(19) United States
(12) Patent Application Publication
(10) Pub. No.: US 2009/0270965 A1
(43) Pub. Date: Oct. 29, 2009

(54) Endovascular Prosthesis for Ascending Aorta

(75) Inventors: Sachin Sinha, Santa Rosa, CA (US); Kristy Peterson, Santa Rosa, CA (US); Janelle Chang, San Francisco, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 12/108,737

(22) Filed: Apr. 24, 2008

(51) Int. Cl. A61F 2/84; A61F 2/82

(52) U.S. Cl. 623/1.11; 623/1.14

(57) ABSTRACT

An endoluminal prosthesis for treating a diseased portion of the ascending aorta includes a tubular graft material having an outer surface and an inner surface and a support structure coupled to the graft material. An anchoring device is coupled to the proximal end of the support structure to engage the sinotubular junction or the sinuses adjacent the sinotubular junction. The anchoring device may be a stent ring with barbs on an outer surface to engage the sinotubular junction, a plurality of anchors extending into the sinuses including hooks to engage the sinuses, a plurality of bent stents with distally facing shoulders extending into the sinuses and engaging a distal edge of the sinuses, or a series of progressively larger diameter stent rings extending into the sinuses to engage the distal edge of the sinuses.

### FIELD OF THE INVENTION

The invention relates to a medical device for use within a body vessel and, in particular, to an endovascular prosthesis for use in the ascending aorta.

### BACKGROUND

The aorta is the major artery that carries blood from the heart to the rest of the body. FIG. 1 is a schematic illustration of the aorta 100 and the heart 108. The aorta 100 includes an ascending aorta 102, an aortic arch 104, and a descending aorta 106. The ascending aorta 102 is the first segment of the aorta 100 where the aorta 100 originates from the heart's left ventricle. Coronary arteries 110 originate at the aortic root 111. The brachiocephalic artery 116, the left common carotid artery 118, and the left subclavian artery 120 branch from the aortic arch 104. The descending artery 106 extends past the diaphragm 112, leading to the abdominal aorta 114.

Aortic dissection occurs when the inner layer of the aorta's artery wall splits open (dissects). This is more likely to occur where pressure on the arterial wall from blood flow is high, such as in the ascending aorta 102. FIG. 2 shows a dissection 122 in the ascending aorta 102. The dissection may be caused by a tear 124 in the aortic wall. When the layers of the aortic wall split open (separate from one another), it creates a false lumen 126 through which pulsatile blood flow can access the inner layers that compose the arterial wall. This tends to cause the split to propagate further. This split may continue distally away from the heart 108 through the aortic arch 104 and down the descending aorta 106 and into its major branches or it may sometimes run proximally back toward the heart 108. Other defects in the wall of the vessel that may be suitable for treatment with the devices and methods described herein are: trans-sections and penetrating ulcers.

The ascending aorta 102 and aortic arch 104 may also be affected by aneurysmal dilatation. The standard surgical approach in patients with ascending aortic aneurysm or dissection involving the aortic root and associated with aortic valve disease is the replacement of the aortic valve and ascending aorta by use of a composite valve graft onto which the two coronary arteries 110 are attached. If the aortic valve leaflets are normal, a valve-sparing aortic root remodeling procedure which keeps the patient's natural valve on site is a reasonable alternative in certain individuals. These open surgical operations rely upon cardiopulmonary bypass, with or without hypothermic circulatory arrest. The associated mortality, morbidity, debility, pain, and expense are all high.

Endovascular methods of reconstruction in the ascending aorta and aortic arch face difficulty in finding healthy vessel tissue on which to land an endovascular prosthesis or stent-graft. As shown in FIGS. 3 and 4, common endovascular prostheses or stent-grafts 150 for use in the descending aorta 106 include a graft material 154, such as woven polymer materials (e.g., Dacron (polyester) or polytetrafluoroethylene (“PTFE”)), and a support structure 152. The support structure 152 expands in the vessel to hold the graft 150 against the vessel wall. The stent-grafts typically have graft material secured onto the inner diameter or outer diameter of the support structure that supports the graft material and/or holds it in place against a vessel wall. The prosthesis is typically secured to a vessel wall upstream and downstream of the aneurysm site spanning the aneurysm with at least one attached expandable annular spring member that provides sufficient radial force so that the prosthesis engages the inner vessel wall of the body lumen to seal the prosthetic lumen from the aneurysm. The spring member needs to be positioned to expand, i.e. land, on a healthy portion of the vessel tissue to hold the prosthesis in place. In a dissection or aneurysm in the ascending aorta, there may not be suitable healthy tissue at one or both ends of the dissection or aneurysm on which to land the spring member.

### SUMMARY OF THE INVENTION

An endoluminal prosthesis for treating a diseased portion of the ascending aorta includes a graft including a tubular graft material and a support structure coupled to the graft material. An anchoring device is coupled to the proximal end of the support structure to engage the sinotubular junction or the sinuses adjacent the sinotubular junction.

In an embodiment, the anchoring device may be a stent ring with barbs on an outer surface thereof coupled to a proximal end of the graft. The prosthesis is delivered to the ascending aorta such that the stent ring is disposed adjacent the sinotubular junction. The prosthesis is expanded such that the stent ring engages the sinotubular junction.

In another embodiment a plurality of anchors are coupled to the proximal end of the graft. The anchors include a hook at the proximal end thereof. The prosthesis is delivered to ascending aorta such that the proximal end of the graft is disposed adjacent the sinotubular junction and the anchors extend into the sinuses. Upon expansion of the prosthesis, the anchors extend outwardly such that the hooks engage the tissue of the sinuses. The prosthesis is pulled distally to secure the engagement of the hooks to the sinuses.

In another embodiment, pluralities of bent or angled stents are coupled to the proximal end of the graft. The bent stents include distally facing shoulders. The prosthesis is delivered to the ascending aorta such that the proximal end of the graft is disposed adjacent the sinotubular junction and the bent stents extend into the sinuses. Upon expansion of the prosthesis, the distally facing shoulders of the bent stents engage a distal edge of the sinuses. The prosthesis is pulled distally to abut the shoulders against the distal edge of the sinuses.

In another embodiment, a series of progressively larger diameter stent rings are coupled to the proximal end of the graft. The prosthesis is delivered to the ascending aorta such that the proximal end of the graft is disposed adjacent the sinotubular junction and the series of stent rings extend into the sinuses. Upon expansion of the prosthesis, the distally facing surfaces of the stent rings engage a distal edge of the sinuses. The prosthesis is pulled distally to abut the stent rings against the distal edge of the sinuses.

### BRIEF DESCRIPTION OF DRAWINGS

The foregoing and other features and advantages of embodiments according to the present invention will be apparent from the following description as illustrated in the accompanying drawings. The accompanying drawings, which are incorporated herein and form a part of the specification, further serve to explain the embodiments and to enable a person skilled in the pertinent art to make and use embodiments thereof. The drawings are not to scale.

FIG. 1 is a schematic illustration of the heart and the aorta.

FIG. 2 is a schematic illustration of the aorta with a dissection.

FIGS. 3 and 4 are schematic illustrations of a prior art endoluminal prosthesis for use in treating an aneurysm in the descending aorta.

FIG. 5 is a cross-sectional illustration of the aortic root and the ascending aorta with a dissection.

FIG. 6 is a schematic illustration of the aortic valve.

FIG. 7 is a schematic side view of an embodiment of an endoluminal prosthesis.

FIG. 8 is a cross-sectional view of the prosthesis of FIG. 7.

FIG. 9 is a schematic cross-sectional view of the prosthesis, shown in FIG. 7, deployed in the ascending aorta.

FIG. 10 is a schematic illustration of another embodiment of a prosthesis.

FIG. 11 is a cross-sectional view of the prosthesis of FIG. 10.

FIG. 12 is a cross-sectional view of the prosthesis of FIG. 10 deployed in the ascending aorta.

FIG. 13 is a schematic illustration of another embodiment of a prosthesis.

FIG. 14 is a cross-sectional view of the prosthesis of FIG. 13.

FIG. 15 is a cross-sectional view of the prosthesis of FIG. 13 deployed in the ascending aorta.

FIG. 16 is a schematic illustration of an alternative embodiment of the prosthesis of FIG. 13.

FIG. 17 is a cross-sectional view of the prosthesis of FIG. 16 deployed in the ascending aorta.

FIG. 18 is a schematic side view of another embodiment of a prosthesis.

FIG. 19 is a schematic cross-sectional view of the prosthesis of FIG. 18.

FIG. 20 is a schematic illustration of the prosthesis of FIG. 18.

FIG. 21 is a cross-sectional view of the prosthesis of FIG. 18 deployed in the ascending aorta.

FIGS. 22-24 are schematic illustrations of the steps of a method of delivering the prosthesis of FIG. 7 to the ascending aorta and deploying the prosthesis therein.

FIGS. 25-27 are schematic illustrations of the steps of a method of delivering the prosthesis of FIG. 10 to the ascending aorta and deploying the prosthesis therein.

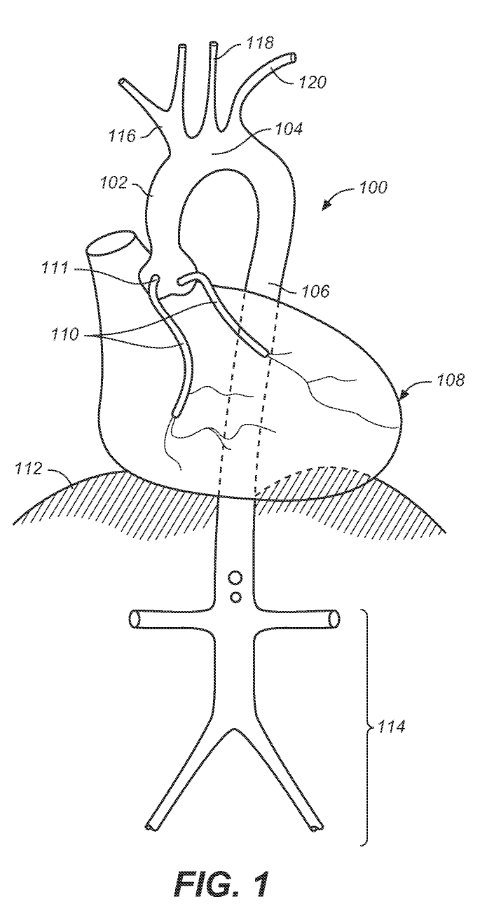

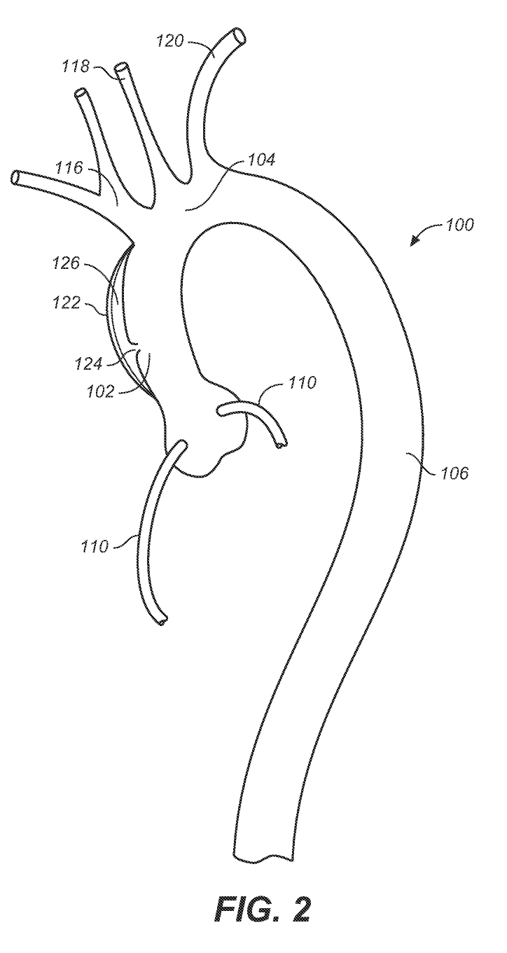

### DETAILED DESCRIPTION

Specific embodiments are now described with reference to the figures, wherein like reference numbers indicate identical or functionally similar elements. The terms “distal” and “proximal” are used for the implanted device in the following description with respect to a position or direction relative to the heart. “Distal” or “distally” are a position distant from or in a direction away from the heart. “Proximal” and “proximally” are a position near or in a direction toward the heart.

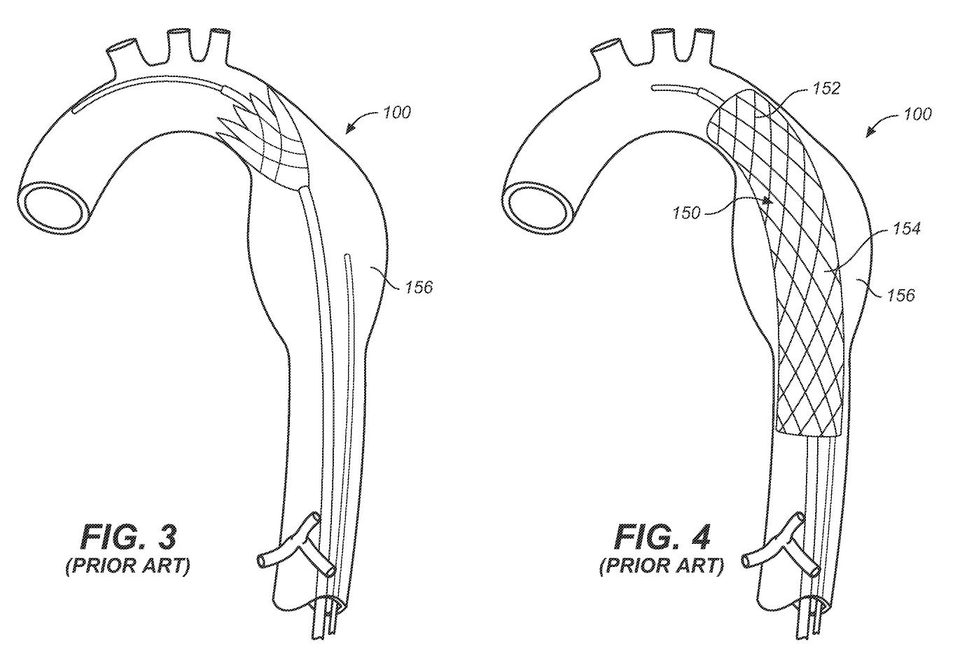

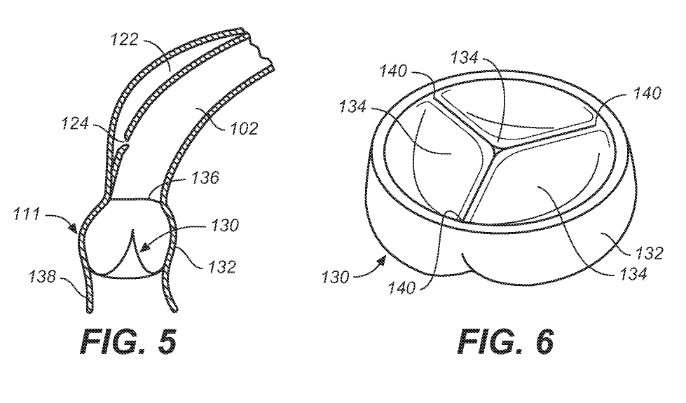

FIG. 5 is a schematic illustration of the junction between the ascending aorta 102 and the heart. The aortic root 111 is the portion of the left ventricular outflow tract which supports the leaflets 134 (shown in FIG. 6) of the aortic valve 130. The aortic root 111 may be delineated by the sinotubular junction 136 distally and the bases of the valve leaflets 134 proximally. The aortic root 111 comprises the sinuses 132, the valve leaflets 134, the commissures 140, and the interleaflet triangles (not shown). The sinotubular junction 136 may provide a suitable landing zone for an endovascular graft in cases where the dissection does not extend into the aortic root, while maintaining an acceptable sinotubular definition (i.e., the relationship between the diameters of the sinuses, the sinotubular junction, and the ascending aorta).

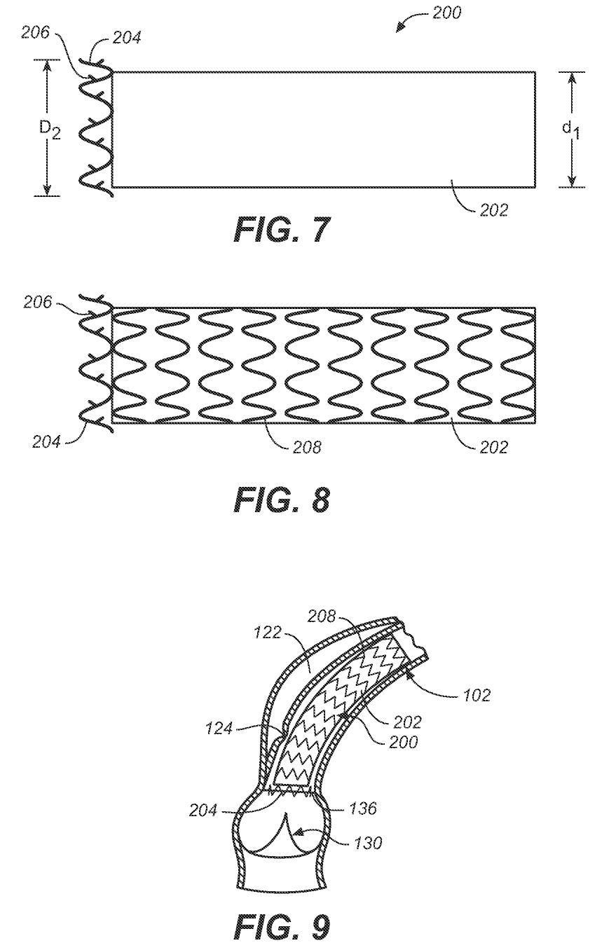

FIG. 7 is a schematic side view of an endovascular prosthesis 200, a stent-graft including a tubular graft material 202 coupled to a series of radially compressible annular support members 208 (FIG. 8). The annular support members 208 support the graft and/or bias the prosthesis 200 into conforming fixed engagement with an interior surface of the ascending aorta 102 (see FIG. 9). The annular support members 208 are ring spring members having predetermined radii and are constructed of a material such as Nitinol in a superelastic, shape set condition. The graft material 202 may be a woven polymer material (e.g., Dacron (polyester) or polytetrafluoroethylene (“PTFE”)) or other suitable graft material known to those of ordinary skill in the art.

A stent ring 204 is coupled to a proximal end of prosthesis 200. Stent ring 204 may include barbs or hooks 206 on an outer surface thereof. Stent ring 204 is preferably made from a shape memory material such as a nickel-titanium alloy (Nitinol). Stent ring 204 is also preferably self-expanding. When prosthesis 200 is expanded such as by releasing it from a sleeve or catheter, an outer surface of graft material 200 defines a first expanded diameter d₁ and an outer surface of stent ring 204 defines a second diameter d₂, wherein second diameter d₂ is 1.05 to 1.1 times larger than first diameter d₁ when prosthesis 200 is not installed. This larger diameter d₂ of stent ring 204 forces barbs 206 to engage the sinotubular junction 136 when prosthesis 200 is installed in the ascending aorta, as shown in FIG. 9. Thus, when installed, stent ring 204 may not have a larger diameter than graft material 202, but the outward expansion force provides a secure attachment to the sinotubular junction.

Stent ring 204 may be coupled to support members 208 and is preferably formed from the same material as support members 208. Stent ring 204 may be formed as a laser cut structure integral with the support members 208 or may be separate from, but be coupled to support members 208 through the graft material or by any other manner known to those of ordinary skill in the art.

FIG. 9 is a schematic cross-sectional view of the prosthesis 200 installed in the ascending aorta 102. Stent ring 204 is expanded at the sinotubular junction 136 such that barbs 206 engage the sinotubular junction. Prosthesis 200 is thus secured in place distally of stent ring 204. Graft material 202 thus covers the tear 124 in the wall of the vessel such that blood does not flow to false lumen 126.

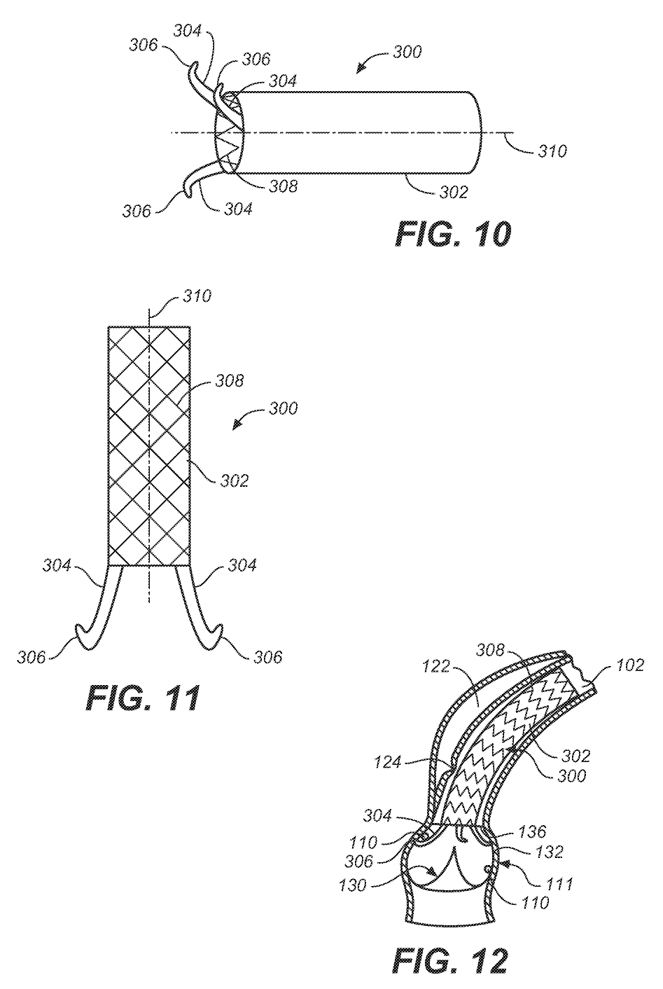

FIGS. 10-12 illustrate a prosthesis 300 that includes a graft material 302 and support members 308 similar to prosthesis 200 described above. Anchors 304 extend from a proximal end of prosthesis 300. In the embodiment shown in FIGS. 10-12, there are three anchors 304 equally spaced around the circumference of the proximal end of prosthesis 300, however, one of ordinary skill in the art would recognize that more or less anchors 304 could be utilized, such as two anchors or four anchors. Each anchor 304 includes a hook or barb 306 at a proximal end thereof. Anchors 304 extend outwardly relative to a longitudinal axis 310 of prosthesis 300.

Anchors 304 are preferably made from the same material as support members 308, for example, shape memory metals and metal alloys such as a nickel-titanium alloy (Nitinol). Anchors 304 may be coupled to support members 308 by any means known to those of ordinary skill in the art. Anchors 304 may be cut from a solid material or may be a looped wire.

When installed in the ascending aorta, as shown in FIG. 12, a proximal end of graft material 302 is disposed adjacent the sinotubular junction. Anchors 304 extend proximally and outwardly from the proximal end of graft material 302 such that hooks 306 engage the sinuses 132, thereby anchoring prosthesis 300 within the ascending aorta 102. Prosthesis 300 is thus secured in place distally of anchors 304 and graft material 302 covers the tear 124 in the wall of the vessel such that blood does not flow into false lumen 126. Anchors 306 are arranged such they do not block the coronary arteries 110.

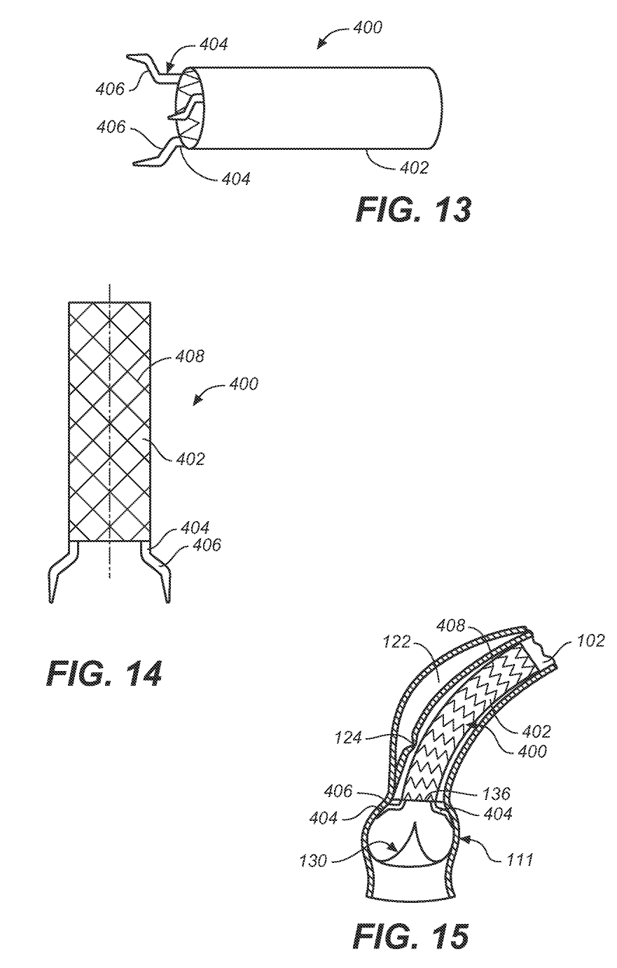

FIGS. 13-15 illustrate a prosthesis 400 that includes a graft material 402 and support members 408 similar to prostheses 200 and 300 described above. Anchors 404 extend from a proximal end of prosthesis 400. Anchors 404 shown in FIGS. 13-15 are bent or angled stents. In particular, anchors 404 bend outwardly such that each anchor includes a distally facing shoulder 406. In the embodiment shown in FIGS. 13-15, there are three anchors 404 equally spaced around the circumference of the proximal end of prosthesis 400, however, one of ordinary skill in the art would recognize that more or less anchors 404 could be utilized, such as two anchors or four anchors.

Anchors 404 are preferably made from the same material as support members 408, for example, shape memory metals and metal alloys such as a nickel-titanium alloy (Nitinol). Anchors 404 may be coupled to support members 408 by any means known to those of ordinary skill in the art.

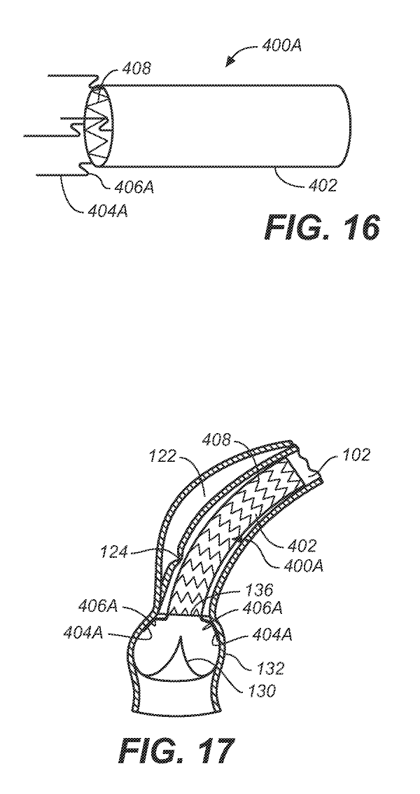

When installed in the ascending aorta, as shown in FIG. 15, a proximal end of graft material 402 is disposed adjacent the sinotubular junction. Anchors 404 extend proximally from the proximal end of graft material 402 such that shoulders 406 abut the inside wall of the sinuses as the sinuses bow outwardly from the sinotubular junction. Proximal of shoulders 406, anchors 404 preferably follow the contours of the sinuses such that anchors 404 do not interfere with blood flow or the operation of the valve leaflets. In the embodiment shown in FIGS. 13-15, anchors 404 have a larger cross-sectional area than in an alternative embodiment of prosthesis 400A shown in FIGS. 16-17 where anchors 404A with shoulders 406A are wires. The larger cross-sectional area of the anchors 404 in the embodiment of FIGS. 13-15 reduces the chance of tissue damage at the sinuses by distributing the contact force between the anchors 404 and the tissue of the sinuses over a larger contact area. This larger area reduces the localized peak force on the tissue and reduces the likelihood that a cheese grating type damage (associated with the interaction between a taut wire and soft tissue) will occur. A larger cross sectional area to reduce the chance of damage to surrounding tissue can also be created by applying a thick coating to wire-type anchors to effectively increase their surface area to reduce the force per unit area imposed on the surrounding tissue.

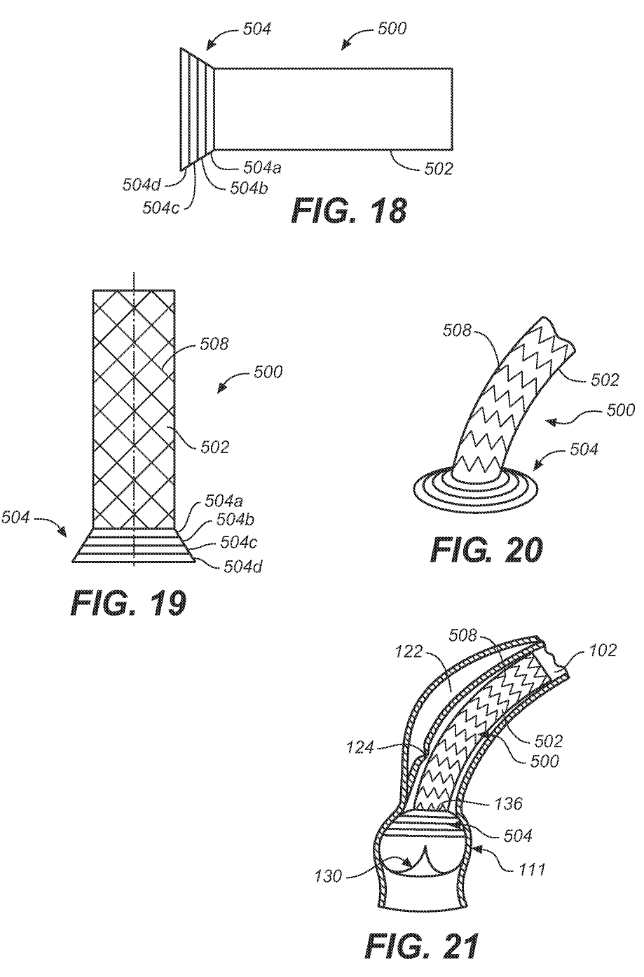

FIGS. 18-21 illustrate a prosthesis 500 that support members 508 similar to prosthesis 200, 300, and 400 described above. A series of stent rings 504 are coupled to a proximal end of prosthesis 500. The embodiment shown in FIGS. 18-21 includes four stent rings 504a, 504b, 504c, and 504d, but one of ordinary skill in the art would recognize that a greater or fewer number of rings could be utilized. Moving proximally, each stent ring 504 has a diameter that is the same or larger than the stent ring immediately distal to the stent ring. Accordingly, stent ring 504a has a diameter that is the same as or larger than the diameter of the proximal end of the prosthesis 500. The diameter of stent ring 504b is the same or larger than the diameter of stent ring 504a; the diameter of stent ring 504c is the same or larger than the diameter of stent ring 504b; and the diameter of stent ring 504d is the same or larger than the diameter of stent ring 504c. While not shown in the figures, once the diameter of the stent rings increasing in size has reached the largest diameter of the sinuses, further stent rings may be sized with reduced diameters to conform to the sinus wall and provide a potentially larger area for fixation and wedging of the device in the anatomy.

Stent rings 504 may be made from the same material as support members 508, for example, shape memory metals and metal alloys such as a nickel-titanium alloy (Nitinol). Stent rings 504 may be coupled to support members 508 and to the next most distal stent ring in the series any means known to those of ordinary skill in the art.

When installed in the ascending aorta, as shown in FIG. 21, a proximal end of graft material 502 is disposed adjacent the sinotubular junction. Stent rings 504 are disposed proximal of the sinotubular junction and abut against the distal edge of the sinuses. The diameters of stent rings 504 are preferably slightly larger than the diameter of the sinus region such that the stent rings press against the sinuses to secure the prosthesis in the ascending aorta. Prosthesis 500 is thus secured in place distally of stent rings 504 and graft material 502 covers tear 124 such that blood does not flow into false lumen 126. As shown here, the stent rings 504 do not extend far enough into the sinus region to block the coronary arteries. However, because of the open frame structure of the stent rings, they could extent into the area of the sinuses to the level of the origin of the coronary arteries, without obstructing blood flow to the origin of those arteries.

Although endovascular prostheses 200, 300, 400, 400A, and 500 have been described as stent-grafts, endovascular prostheses 200, 300, 400, 400A, and 500 may be any prosthetic device for use in a vessel, in particular, the ascending aorta. Further, the securing mechanisms, stent ring 204, anchors 304, 404, or 404A, or stent ring series 504, either alone or in combination with all or a portion of prosthesis 200, 300, 400, 400A, or 500, may be used as a docking station to which other devices may be coupled.

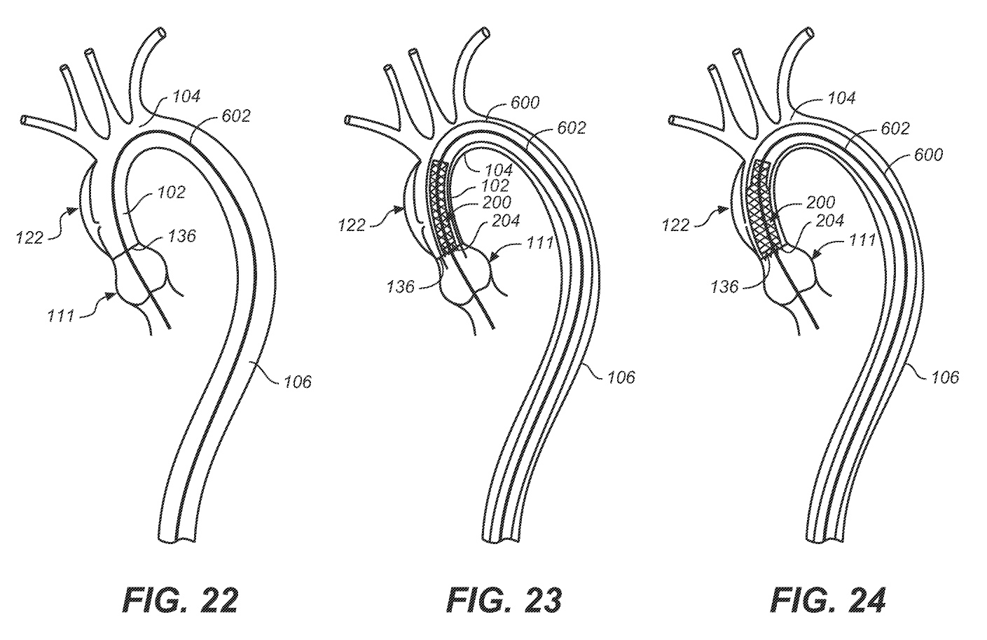

FIGS. 22-24 diagrammatically illustrate delivering prosthesis 200 to the ascending aorta 102 and deploying prosthesis 200. A guidewire 602 is tracked through the descending aorta 106, aortic arch 104, ascending aorta 102, and into the aortic root 111, as illustrated in FIG. 22. Access can be gained through the femoral artery or other locations as would be understood by those of ordinary skill in the art. A catheter 600 with prosthesis 200 enclosed therein is tracked along guidewire 602 to the aortic root 111, as shown in FIG. 23. Catheter 600 is shown diagrammatically and may include several components known to those of ordinary skill in the art, such as inner and outer tubes, a sheath, and a stop. After catheter 600 is in place and prosthesis 200 is aligned such that stent ring 204 is aligned with the sinotubular junction 136, catheter 600 is withdrawn distally (away from the heart) to release prosthesis 200, as shown in FIG. 24. Hooks or barbs 206 on stent ring 204 engage the sinotubular junction. After catheter 600 is completely withdrawn and guidewire 602 is removed, prosthesis 200 is deployed within ascending aorta 102 as shown in FIG. 9.

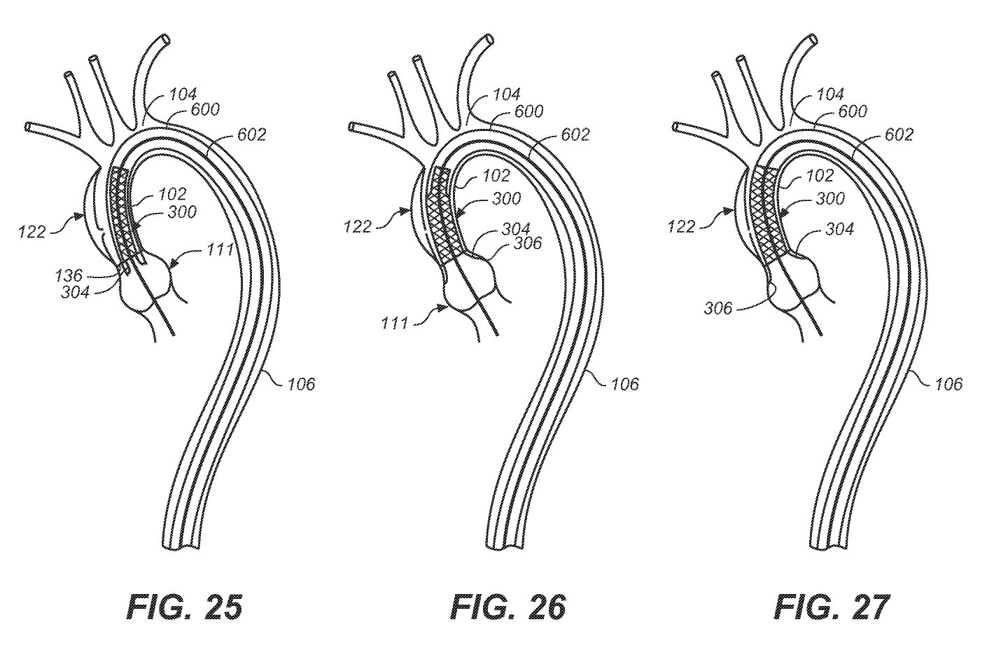

FIGS. 25-27 diagrammatically illustrate delivering prosthesis 300 to the ascending aorta 102 and deploying prosthesis 300. A guidewire 602 is tracked through the descending aorta 106, aortic arch 104, ascending aorta 102, and into the aortic root 111, as shown in FIG. 25. Access can be gained through the femoral artery or other locations as would be understood by those of ordinary skill in the art. A catheter 600 with prosthesis 300 enclosed therein is tracked along guidewire 602 to the aortic root 111, as shown in FIG. 26. Catheter 600 is shown diagrammatically and may include several components known to those of ordinary skill in the art, such as inner and outer tubes, a sheath, and a stop. In particular, with respect to this embodiment, catheter 600 may include a device to pull prosthesis 300 distally, as described in more detail below. After catheter 600 is in place and prosthesis 300 is aligned such that the proximal end of graft material 302 is aligned with the sinotubular junction 135, withdrawal of the sheath of the catheter 600 is begun to begin to release prosthesis 300. As the sheath is withdrawn, anchors 304 are released within the aortic root 111 and hooks 306 penetrate tissue of the sinuses 132. After the proximal end of the prosthesis 300 is deployed the catheter 600, with a portion of the prosthesis still undeployed, prosthesis 300 is pulled back to aid in fixation of hooks 306 in the sinus tissue, as shown in FIG. 27. After the sheath is fully retracted and the catheter 600 is completely withdrawn and guidewire 602 is removed, prosthesis 300 remains deployed within ascending aorta 102 as shown in FIG. 12.

The method described with respect to FIGS. 25-27 may be used to deliver and deploy prostheses 400, 400A, and 500. With respect to prostheses 400 and 400A, the prostheses are pulled distally such that shoulders 406 or 406A abut the distal edge of the sinuses. Similarly, with respect to prosthesis 500, prosthesis 500 is pulled distally such that the series of stent rings 504 abut the distal edge of the sinuses.

Although prostheses of the embodiments described above have been shown extending only within the ascending aorta 102, it would be understood by those of ordinary skill in the art that the prosthesis may extending into the aortic arch 104 and into the descending aorta 106, if necessary. In such a situation, the prosthesis must accommodate branches 116, 118, 120, which can be done by means known to those of ordinary skill in the art. For example, openings may be provided in the graft material and the prosthesis may be oriented such that the openings align with branches 115, 118, and 120, as explained in U.S. Published Patent Application Publication No. 2007/0233229, which is incorporated by reference herein in its entirety. Alternatively, a modular system may be provided as explained in U.S. Pat. No. 6,814,752, which is incorporated by reference herein in its entirety.

While various embodiments according to the present invention have been described above, it should be understood that they have been presented by way of illustration and example only, and not limitation. It will be apparent to persons skilled in the relevant art that various changes in form and detail can be made therein without departing from the spirit and scope thereof. It will also be understood that each feature of each embodiment discussed herein, and of each reference cited herein, can be used in combination with the features of any other embodiment. All patents and publications discussed herein are incorporated by reference herein in their entirety.

What is claimed is:

1. An endoluminal prosthesis comprising:
a tubular graft material having an outer surface and an inner surface, wherein the outer surface defines a first diameter;
a support structure coupled to the graft material; and
a plurality of anchors, each anchor having a distal end coupled to a proximal end of the support structure, a proximal end opposite the distal end, and a hook extending outwardly from the proximal end, wherein the anchors extend outwardly such that a second diameter defined by the proximal ends of the anchors is larger than the first diameter.

2. The prosthesis of claim 1, wherein the prosthesis includes three anchors.

3. The prosthesis of claim 1, wherein the prosthesis includes two anchors.

4. An endoluminal prosthesis comprising:
a tubular graft material having an outer surface and an inner surface, wherein the outer surface defines a first diameter;
a support structure coupled to the graft material; and
a stent ring coupled to a proximal end of the support structure, wherein an outer surface of the stent ring defines a second diameter that is larger than the first diameter, and wherein a plurality of barbs or hooks are coupled to the outer surface of the stent ring.

5. The endoluminal prosthesis of claim 4, wherein the second diameter is 1.05 to 1.1 times larger than the first diameter.

6. An endoluminal prosthesis comprising:
a tubular graft material having an outer surface and an inner surface, wherein the outer surface defines a first diameter;
a support structure coupled to the graft material; and
a plurality of stent rings coupled to a proximal end of the support structure, wherein a first stent ring coupled to the support structure has a second diameter that is larger than the first diameter, and wherein each stent ring proximal of the first stent ring has a diameter equal to or larger than the stent ring distally adjacent thereto.

7. The endoluminal prosthesis of claim 6, wherein the plurality of stent rings includes two stent rings.

8. The endoluminal prosthesis of claim 7, wherein the plurality of stent rings includes four stent rings.

9. An endoluminal prosthesis comprising:
a tubular graft material having an outer surface and an inner surface, wherein the outer surface defines a first diameter;
a support structure coupled to the graft material; and
a plurality of angled stents extending longitudinally from and coupled to a proximal end of the support structure, wherein each stent includes a distally facing shoulder, wherein a second diameter defined by an outer edge of each shoulder is larger than the first diameter.

10. The endoluminal of claim 9, wherein the plurality of angled stents includes two stents.

11. The endoluminal prosthesis of claim 9, wherein the plurality of angled stents includes four stents.

12. A method for treating a diseased portion of the ascending aorta comprising the steps of:
delivering a prosthesis to the ascending aorta, wherein the prosthesis includes a graft and a stent ring coupled to a proximal end of the graft, wherein the stent ring includes an outer surface having a plurality of barbs disposed thereon;
aligning the prosthesis such that the stent ring is disposed adjacent the sinotubular junction between the aortic sinuses and the ascending aorta;
deploying the prosthesis such that the barbs on the stent ring engage the sinotubular junction.

13. The method of claim 12, wherein the graft includes a tubular graft material having an outer surface and an inner surface and a support structure coupled to the graft material, wherein the stent ring is coupled to a proximal end of the support structure.

14. The method of claim 13, wherein the step of deploying the prosthesis further includes the expanding the support structure and the graft material to contact an inner surface of the ascending aorta.

15. The method of claim 12, wherein the diseased portion of the ascending aorta includes a dissection.

16. The method of claim 12, wherein the diseased portion of the ascending aorta includes an aneurysm.

17. A method for treating a diseased portion of the ascending aorta comprising the steps of:
delivering a prosthesis to the ascending aorta, wherein the prosthesis includes a graft and a plurality of anchors coupled to and extending proximally from a proximal end of the graft, and wherein a proximal end of each anchor includes a hook extending outwardly;
aligning the prosthesis such that a proximal end of the graft is disposed adjacent the sinotubular junction and the plurality of anchors extend into the region of the aortic sinuses;
deploying the prosthesis such that the anchors expand outwardly such that the hooks engage tissue of the sinuses.

18. The method of claim 17, wherein the step of deploying the prosthesis further includes the step of moving the prosthesis distally after the anchors have expanded outwardly such that the hooks engage tissue of the sinuses.

19. The method of claim 17, wherein the graft includes a tubular graft material having an outer surface and an inner surface and a support structure coupled to the graft material, wherein the anchors are coupled to a proximal end of the support structure.

20. The method of claim 19, wherein the step of deploying the prosthesis further includes expanding the support structure and the graft material to contact an inner surface of the ascending aorta.

21. The method of claim 17, wherein the diseased portion of the ascending aorta includes a dissection.

22. The method of claim 17, wherein the diseased portion of the ascending aorta includes an aneurysm.

23. A method for treating a diseased portion of the ascending aorta comprising the steps of:
delivering a prosthesis to the ascending aorta, wherein the prosthesis includes a graft and a plurality of anchors coupled to and extending proximally from a proximal end of the graft, and wherein a distal portion of each anchor is bent such that the anchor includes a distally facing shoulder;
aligning the prosthesis such that a proximal end of the graft is disposed adjacent the sinotubular junction and the plurality of anchors extend into the region of the aortic sinuses;
deploying the prosthesis such that the anchors expand outwardly such that distally facing shoulders of the anchors engage the distal edge of the sinuses.

24. The method of claim 23, wherein the step of deploying the prosthesis further includes the step of moving the prosthesis distally after the anchors have expanded outwardly such that the distally facing shoulders engage the distal edge of the sinuses.

25. The method of claim 23, wherein the graft includes a tubular graft material having an outer surface and an inner surface and a support structure coupled to the graft material, wherein the anchors are coupled to a proximal end of the support structure.

26. The method of claim 25, wherein the step of deploying the prosthesis further includes expanding the support structure and the graft material to contact an inner surface of the ascending aorta.

27. The method of claim 23, wherein the diseased portion of the ascending aorta includes a dissection.

28. The method of claim 23, wherein the diseased portion of the ascending aorta includes an aneurysm.

29. A method for treating a diseased portion of the ascending aorta comprising the steps of:
delivering a prosthesis to the ascending aorta, wherein the prosthesis includes a graft and a plurality of stent rings coupled to a proximal end of the graft, wherein each stent ring has a diameter larger than the diameter of the stent ring distally adjacent thereto;
aligning the prosthesis such that a proximal end of the graft is disposed adjacent the sinotubular junction and the plurality of stent rings extend into the region of the aortic sinuses;
deploying the prosthesis such that the stent rings anchors expand outwardly and engage the distal edge of the sinuses.

30. The method of claim 29, wherein the step of deploying the prosthesis further includes the step of moving the prosthesis distally after the stent rings have expanded have expanded outwardly such that the stent rings engage the distal edge of the sinuses.

31. The method of claim 29, wherein the graft includes a tubular graft material having an outer surface and an inner surface and a support structure coupled to the graft material, wherein the distal most stent ring is coupled to a proximal end of the support structure.

32. The method of claim 31, wherein the step of deploying the prosthesis further includes expanding the support structure and the graft material to contact an inner surface of the ascending aorta.

33. The method of claim 29, wherein the diseased portion of the ascending aorta includes a dissection.

34. The method of claim 29, wherein the diseased portion of the ascending aorta includes an aneurysm.
